(19) United States
(12) Patent Application Publication
(10) Pub. No.: US 2023/0037787 A1
(43) Pub. Date: Feb. 9, 2023

(54) METHODS AND COMPOSITIONS RELATING TO HOT-START, ONE-STEP, REVERSE TRANSCRIPTION-COUPLED PCR

(71) Applicant: Wayne State University, Detroit, MI (US)

(72) Inventor: Jian-Ping Jin, Chicago, IL (US)

(21) Appl. No.: 17/862,825

(22) Filed: Jul. 12, 2022

Related U.S. Application Data
(60) Provisional application No. 63/220,631, filed on Jul. 12, 2021.

(51) Int. Cl. C12P 19/34; C12Q 1/6844; C12N 9/12

(52) U.S. Cl. CPC C12P 19/34; C12Q 1/6844; C12N 9/1252; C12Y 207/07007

(57) ABSTRACT

Methods of amplifying an RNA template according to aspects of the present disclosure include: providing a composition including a recombinant thermostable DNA polymerase including SEQ ID NO:1, or a variant thereof having at least 99% identity to SEQ ID NO:1; and a reaction buffer, the reaction buffer including 10-30 mM Tris-HCl, pH 8.5-9.0; 20-40 mM KCl; 5-15 mM (NH₄)₂SO₄; 1.5-3.5 mM Mn²⁺; 0.01-0.20% (w/v) gelatin and/or serum albumin; 0.05-0.15% (w/v) of a nonionic detergent; with the proviso that no more than 0.1 mM of Mg²⁺ is present in the composition; and, optionally, 0.01-0.1 mM of a chelating agent is present in the composition.

### REFERENCE TO RELATED APPLICATION

This application claims priority from U.S. Provisional Patent Application Ser. No. 63/220,631, filed Jul. 12, 2021, the entire content of which is incorporated herein by reference.

### FIELD OF THE INVENTION

Methods and compositions relating to hot-start, one-step, reverse transcription-coupled polymerase chain reaction (PCR) according to general aspects of the present disclosure are described herein. According to specific aspects, methods and compositions relating to hot-start, one-step, reverse transcription-coupled polymerase chain reaction (PCR) including SEQ ID NO:1, or a variant thereof having at least 99% identity to SEQ ID NO:1 are described herein.

### BACKGROUND OF THE INVENTION

Conventional methods of producing amplified copies of an RNA template require two enzymes: a thermosensitive reverse transcriptase (RT) to convert RNA into cDNA, and 2) a thermostable DNA polymerase, such as Taq DNA polymerase, to perform polymerase chain reaction (PCR). Such two-enzyme methods require the start of RT reaction at a temperature less than about 50-55° C. and that creates at least two disadvantages: a) ineffective unfolding of the target RNA templates, which reduces sensitivity; and b) lower specificity and target sensitivity due to potential mismatch of the RT primer.

There is a continuing need for improved compositions and methods for amplification of nucleic acid, especially RNA, targets.

### SUMMARY OF THE INVENTION

Compositions according to aspects of the present disclosure include a recombinant thermostable DNA polymerase including SEQ ID NO:1, or a variant thereof having at least 99% identity to SEQ ID NO:1; and a reaction buffer, the reaction buffer including 10-30 mM Tris-HCl, pH 8.5-9.0; 20-40 mM KCl; 5-15 mM (NH₄)₂SO₄; 1.5-3.5 mM Mn²⁺; 0.01-0.20% (w/v) gelatin and/or serum albumin; and 0.05-0.15% (w/v) of a nonionic detergent, with the proviso that no more than 0.1 mM Mg²⁺ is present in the composition. According to aspects of the present disclosure, the nonionic detergent is polysorbate 20. According to aspects of the present disclosure, the reaction buffer includes dNTPs. According to aspects of the present disclosure, the composition is liquid, frozen or lyophilized. According to aspects of the present disclosure, the composition is stored at a temperature in the range of −80° C. to 30° C.

Compositions according to aspects of the present disclosure include a recombinant thermostable DNA polymerase including SEQ ID NO:1, or a variant thereof having at least 99% identity to SEQ ID NO:1; and a reaction buffer, the reaction buffer including 10-30 mM Tris-HCl, pH 8.5-9.0; 20-40 mM KCl; 5-15 mM (NH₄)2SO4; 1.5-3.5 mM Mn²⁺; 0.01-0.20% (w/v) gelatin and/or serum albumin; 0.05-0.15% (w/v) of a nonionic detergent; and a chelating agent.

According to aspects of the present disclosure, the chelating agent is present in a concentration of 0.01-0.1 mM. According to aspects of the present disclosure, the nonionic detergent is polysorbate 20. According to aspects of the present disclosure, the reaction buffer includes dNTPs. According to aspects of the present disclosure, the composition is liquid, frozen or lyophilized. According to aspects of the present disclosure, the composition is stored at a temperature in the range of −80° C. to 30° C.

Compositions according to aspects of the present disclosure include a recombinant thermostable DNA polymerase including SEQ ID NO:1, or a variant thereof having at least 99% identity to SEQ ID NO:1; and a reaction buffer, the reaction buffer including 10-30 mM Tris-HCl, pH 8.5-9.0; 20-40 mM KCl; 5-15 mM (NH₄)₂SO₄; 1.5-3.5 mM Mn²⁺; 0.01-0.20% (w/v) gelatin and/or serum albumin; 0.05-0.15% (w/v) of a nonionic detergent; and a chelating agent selected from the group consisting of: N-(2-hydroxyethyl)ethylenediaminetriacetic acid (HEDTA), ethylenediaminetetraacetic acid (EDTA), ethyleneglycol bis(2-aminoethyl ether)-N,N,N′,N′ tetraacetic acid (EGTA), diethylenetriaminepentaacetic acid (DTPA), trans-1,2-diaminocyclohexane-N,N,N′,N′-tetraacetic acid (CDTA), nitrilotriacetic acid (NTA), and a combination of any two or more thereof. According to aspects of the present disclosure, the chelating agent is present in a concentration of 0.01-0.1 mM.

According to aspects of the present disclosure, the nonionic detergent is polysorbate 20. According to aspects of the present disclosure, the reaction buffer includes dNTPs. According to aspects of the present disclosure, the composition is liquid, frozen or lyophilized. According to aspects of the present disclosure, the composition is stored at a temperature in the range of −80° C. to 30° C.

Compositions according to aspects of the present disclosure include a recombinant thermostable DNA polymerase including SEQ ID NO:1, or a variant thereof having at least 99% identity to SEQ ID NO:1; and a reaction buffer, the reaction buffer including 10-30 mM Tris-HCl, pH 8.5-9.0; 20-40 mM KCl; 5-15 mM (NH₄)₂SO₄; 1.5-3.5 mM Mn²⁺; 0.01-0.20% (w/v) gelatin and/or serum albumin; 0.05-0.15% (w/v) of a nonionic detergent; and a chelating agent.

According to aspects of the present disclosure, the chelating agent is present in a concentration of 0.01-0.1 mM. According to aspects of the present disclosure, the nonionic detergent is polysorbate 20. According to aspects of the present disclosure, the reaction buffer includes dNTPs. According to aspects of the present disclosure, the composition is liquid, frozen or lyophilized. According to aspects of the present disclosure, the composition is stored at a temperature in the range of −80° C. to 30° C.

According to aspects of the present disclosure, the reaction buffer includes 25 mM Tris-HCl, pH 8.8; 30 mM KCl; 10 mM (NH₄)₂SO₄; 1.5-3.5 mM Mn²⁺; 0.1% (w/v) gelatin and/or serum albumin; and 0.1% (w/v) of a nonionic detergent. According to aspects of the present disclosure, the nonionic detergent is polysorbate 20. According to aspects of the present disclosure, the reaction buffer includes dNTPs. According to aspects of the present disclosure, the composition is liquid, frozen or lyophilized. According to aspects of the present disclosure, the composition is stored at a temperature in the range of −80° C. to 30° C.

According to aspects of the present disclosure, the reaction buffer includes 25 mM Tris-HCl, pH 8.8; 30 mM KCl; 10 mM (NH₄)₂SO₄; 1.5-3.5 mM Mn²⁺; 0.1% (w/v) gelatin and/or serum albumin; 0.1% (w/v) of a nonionic detergent; and a chelating agent.

According to aspects of the present disclosure, the chelating agent is selected from the group consisting of: N-(2-hydroxyethyl)ethylenediaminetriacetic acid (HEDTA), ethylenediaminetetraacetic acid (EDTA), ethyleneglycol bis(2-aminoethyl ether)-N,N,N′,N′ tetraacetic acid (EGTA), diethylenetriaminepentaacetic acid (DTPA), trans-1,2-diaminocyclohexane-N,N,N′,N′-tetraacetic acid (CDTA), nitrilotriacetic acid (NTA), and a combination of any two or more thereof. According to aspects of the present disclosure, the chelating agent is present in a concentration of 0.01-0.1 mM. According to aspects of the present disclosure, the nonionic detergent is polysorbate 20. According to aspects of the present disclosure, the reaction buffer includes dNTPs. According to aspects of the present disclosure, the composition is liquid, frozen or lyophilized. According to aspects of the present disclosure, the composition is stored at a temperature in the range of −80° C. to 30° C.

According to aspects of the present disclosure, the reaction buffer includes 25 mM Tris-HCl, pH 8.8; 30 mM KCl; 10 mM (NH₄)₂SO₄; 1.5-3.5 mM Mn²⁺; 0.1% (w/v) gelatin and/or serum albumin; 0.1% (w/v) of a nonionic detergent; and a chelating agent.

According to aspects of the present disclosure, the chelating agent is HEDTA. According to aspects of the present disclosure, the chelating agent is present in a concentration of 0.01-0.1 mM. According to aspects of the present disclosure, the nonionic detergent is polysorbate 20. According to aspects of the present disclosure, the reaction buffer includes dNTPs. According to aspects of the present disclosure, the composition is liquid, frozen or lyophilized. According to aspects of the present disclosure, the composition is stored at a temperature in the range of −80° C. to 30° C.

Methods of amplifying an RNA template according to aspects of the present disclosure include: providing a composition including a recombinant thermostable DNA polymerase including SEQ ID NO:1, or a variant thereof having at least 99% identity to SEQ ID NO:1; and a reaction buffer, the reaction buffer including 10-30 mM Tris-HCl, pH 8.5-9.0; 20-40 mM KCl; 5-15 mM (NH₄)₂SO₄; 1.5-3.5 mM Mn²⁺; 0.01-0.20% (w/v) gelatin and/or serum albumin; and 0.05-0.15% (w/v) of a nonionic detergent, with the proviso that no more than 0.1 mM Mg²⁺ is present in the composition; adding an RNA template, a reverse transcription primer, and a pair of amplification primers, of which one can be identical to the reverse transcription primer, to the composition in a container, producing a reaction mixture in the container; incubating the reaction mixture in the container at a denaturing temperature in the range of 92° C. to 97° C. for 0.5 to 5 minutes, producing denatured RNA template in the reaction mixture in the container; incubating the reaction mixture in the container at a reverse transcription temperature in the range of 70° C. to 77° C. for 1 to 20 minutes, producing cDNA in the reaction mixture in the container; and amplifying the cDNA in the reaction mixture in the container by an amplification reaction.

Methods of amplifying an RNA template according to aspects of the present disclosure include: providing a composition including a recombinant thermostable DNA polymerase including SEQ ID NO:1, or a variant thereof having at least 99% identity to SEQ ID NO:1; and a reaction buffer, the reaction buffer including 10-30 mM Tris-HCl, pH 8.5-9.0; 20-40 mM KCl; 5-15 mM (NH₄)₂SO₄; 1.5-3.5 mM Mn²⁺; 0.01-0.20% (w/v) gelatin and/or serum albumin; 0.05-0.15% (w/v) of a nonionic detergent; and 0.01-0.1 mM of a chelating agent; adding an RNA template, a reverse transcription primer, and a pair of amplification primers, of which one can be identical to the reverse transcription primer, to the composition in a container, producing a reaction mixture in the container; incubating the reaction mixture in the container at a denaturing temperature in the range of 92° C. to 97° C. for 0.5 to 5 minutes, producing denatured RNA template in the reaction mixture in the container; incubating the reaction mixture in the container at a reverse transcription temperature in the range of 70° C. to 77° C. for 1 to 20 minutes, producing cDNA in the reaction mixture in the container; and amplifying the cDNA in the reaction mixture in the container by an amplification reaction.

Methods of amplifying an RNA template according to aspects of the present disclosure include: providing a composition including a recombinant thermostable DNA polymerase including SEQ ID NO:1, or a variant thereof having at least 99% identity to SEQ ID NO:1; and a reaction buffer, the reaction buffer including 10-30 mM Tris-HCl, pH 8.5-9.0; 20-40 mM KCl; 5-15 mM (NH₄)2SO4; 1.5-3.5 mM Mn²⁺; 0.01-0.20% (w/v) gelatin and/or serum albumin; 0.05-0.15% (w/v) of a nonionic detergent; and 0.01-0.1 mM of a chelating agent selected from the group consisting of: N-(2-hydroxyethyl)ethylenediaminetriacetic acid (HEDTA), ethylenediaminetetraacetic acid (EDTA), ethyleneglycol bis(2-aminoethyl ether)-N,N,N′,N′ tetraacetic acid (EGTA), diethylenetriaminepentaacetic acid (DTPA), trans-1,2-diaminocyclohexane-N,N,N′,N′-tetraacetic acid (CDTA), nitrilotriacetic acid (NTA), and a combination of any two or more thereof; adding an RNA template, a reverse transcription primer, and a pair of amplification primers, of which one can be identical to the reverse transcription primer, to the composition in a container, producing a reaction mixture in the container; incubating the reaction mixture in the container at a denaturing temperature in the range of 92° C. to 97° C. for 0.5 to 5 minutes, producing denatured RNA template in the reaction mixture in the container; incubating the reaction mixture in the container at a reverse transcription temperature in the range of 70° C. to 77° C. for 1 to 20 minutes, producing cDNA in the reaction mixture in the container; and amplifying the cDNA in the reaction mixture in the container by an amplification reaction. According to aspects of the present disclosure, the chelating agent is present in a concentration of 0.01-0.1 mM.

Methods of amplifying an RNA template according to aspects of the present disclosure include: providing a composition including a recombinant thermostable DNA polymerase including SEQ ID NO:1, or a variant thereof having at least 99% identity to SEQ ID NO:1; and a reaction buffer, the reaction buffer including 10-30 mM Tris-HCl, pH 8.5-9.0; 20-40 mM KCl; 5-15 mM (NH₄)₂SO₄; 1.5-3.5 mM Mn²⁺; 0.01-0.20% (w/v) gelatin and/or serum albumin; 0.05-0.15% (w/v) of a nonionic detergent; and 0.01-0.1 mM of a chelating agent. According to aspects of the present disclosure, the chelating agent is HEDTA.

Methods of amplifying an RNA template according to aspects of the present disclosure include: providing a composition including a recombinant thermostable DNA polymerase including SEQ ID NO:1, or a variant thereof having at least 99% identity to SEQ ID NO:1; and a reaction buffer, the reaction buffer including 25 mM Tris-HCl, pH 8.8; 30 mM KCl; 10 mM (NH₄)₂SO₄; 1.5-3.5 mM Mn²⁺; 0.1% (w/v) gelatin and/or serum albumin; and 0.1% (w/v) of a nonionic detergent, with the proviso that no more than 0.1 mM Mg²⁺ is present in the composition; adding an RNA template, a reverse transcription primer, and a pair of amplification primers, of which one can be identical to the reverse transcription primer, to the composition in a container, producing a reaction mixture in the container; incubating the reaction mixture in the container at a denaturing temperature in the range of 92° C. to 97° C. for 0.5 to 5 minutes, producing denatured RNA template in the reaction mixture in the container; incubating the reaction mixture in the container at a reverse transcription temperature in the range of 70° C. to 77° C. for 1 to 20 minutes, producing cDNA in the reaction mixture in the container; and amplifying the cDNA in the reaction mixture in the container by an amplification reaction.

Methods of amplifying an RNA template according to aspects of the present disclosure include: providing a composition including a recombinant thermostable DNA polymerase including SEQ ID NO:1, or a variant thereof having at least 99% identity to SEQ ID NO:1; and a reaction buffer, the reaction buffer including 25 mM Tris-HCl, pH 8.8; 30 mM KCl; 10 mM (NH₄)₂SO₄; 1.5-3.5 mM Mn²⁺; 0.1% (w/v) gelatin and/or serum albumin; 0.1% (w/v) of a nonionic detergent; and 0.01-0.1 mM of a chelating agent; adding an RNA template, a reverse transcription primer, and a pair of amplification primers, of which one can be identical to the reverse transcription primer, to the composition in a container, producing a reaction mixture in the container; incubating the reaction mixture in the container at a denaturing temperature in the range of 92° C. to 97° C. for 0.5 to 5 minutes, producing denatured RNA template in the reaction mixture in the container; incubating the reaction mixture in the container at a reverse transcription temperature in the range of 70° C. to 77° C. for 1 to 20 minutes, producing cDNA in the reaction mixture in the container; and amplifying the cDNA in the reaction mixture in the container by an amplification reaction.

Methods of amplifying an RNA template according to aspects of the present disclosure include: providing a composition including a recombinant thermostable DNA polymerase including SEQ ID NO:1, or a variant thereof having at least 99% identity to SEQ ID NO:1; and a reaction buffer, the reaction buffer including 25 mM Tris-HCl, pH 8.8; 30 mM KCl; 10 mM (NH₄)₂SO₄; 1.5-3.5 mM Mn²⁺; 0.1% (w/v) gelatin and/or serum albumin; 0.1% (w/v) of a nonionic detergent; and 0.01-0.1 mM of a chelating agent selected from the group consisting of: N-(2-hydroxyethyl)ethylenediaminetriacetic acid (HEDTA), ethylene diaminetetraacetic acid (EDTA), ethyleneglycol bis(2-aminoethyl ether)-N,N,N′,N′ tetraacetic acid (EGTA), diethylenetriaminepentaacetic acid (DTPA), trans-1,2-diaminocyclohexane-N,N,N′,N′-tetraacetic acid (CDTA), nitrilotriacetic acid (NTA), and a combination of any two or more thereof; adding an RNA template, a reverse transcription primer, and a pair of amplification primers, of which one can be identical to the reverse transcription primer, to the composition in a container, producing a reaction mixture in the container; incubating the reaction mixture in the container at a denaturing temperature in the range of 92° C. to 97° C. for 0.5 to 5 minutes, producing denatured RNA template in the reaction mixture in the container; incubating the reaction mixture in the container at a reverse transcription temperature in the range of 70° C. to 77° C. for 1 to 20 minutes, producing cDNA in the reaction mixture in the container; and amplifying the cDNA in the reaction mixture in the container by an amplification reaction. According to aspects of the present disclosure, the chelating agent is present in a concentration of 0.01-0.1 mM.

Methods of amplifying an RNA template according to aspects of the present disclosure include: providing a composition including a recombinant thermostable DNA polymerase including SEQ ID NO:1, or a variant thereof having at least 99% identity to SEQ ID NO:1; and a reaction buffer, the reaction buffer including 25 mM Tris-HCl, pH 8.8; 30 mM KCl; 10 mM (NH₄)₂SO₄; 1.5-3.5 mM Mn²⁺; 0.1% (w/v) gelatin and/or serum albumin; 0.1% (w/v) of a nonionic detergent; and 0.01-0.1 mM of a chelating agent, According to aspects of the present disclosure, the chelating agent is HEDTA.

### BRIEF DESCRIPTION OF THE DRAWINGS

FIG. 1 is an image of an agarose gel electrophoresis result showing the final yield of DNA amplified from various copies, 1−1×10⁴ of synthetic SARS-CoV-2 RNA standard;

FIG. 2A is an image of an agarose gel showing that the DNA polymerase of SEQ ID NO:1 gives effective amplification of an RNA target in the presence of 2 mM Mn²⁺ and the presence of Mg²⁺ diminished the polymerase activity;

FIG. 2B is an image of an agarose gel showing that in a control standard PCR using Taq polymerase and DNA template, the presence of Mg²⁺ but not Mn²⁺ gives the expected polymerase activity; and

FIGS. 3A and 3B are images of agarose gels showing that various concentrations of Mn²⁺ are used for effective amplification using different primer pairs and/or targeting sequences for the amplifications of SARS-CoV2 RNA targets using the DNA polymerase of SEQ ID NO:1.

### DETAILED DESCRIPTION OF THE INVENTION

Scientific and technical terms used herein are intended to have the meanings commonly understood by those of ordinary skill in the art. Such terms are found defined and used in context in various standard references illustratively including J. Sambrook and D. W. Russell, Molecular Cloning: A Laboratory Manual, Cold Spring Harbor Laboratory Press; 3rd Ed., 2001; F. M. Ausubel, Ed., Short Protocols in Molecular Biology, Current Protocols; 5th Ed., 2002; B. Alberts et al., Molecular Biology of the Cell, 4th Ed., Garland, 2002; D. L. Nelson and M. M. Cox, Lehninger Principles of Biochemistry, 4th Ed., W. H. Freeman & Company, 2004; Herdewijn, P. (Ed.), Oligonucleotide Synthesis: Methods and Applications, Methods in Molecular Biology, Humana Press, 2004; C. W. Dieffenbach et al., PCR Primer: A Laboratory Manual, Cold Spring Harbor Laboratory Press, 2003; and V. Demidov et al., DNA Amplification: Current Technologies and Applications, Taylor & Francis, 2004.

The singular terms “a,” “an,” and “the” are not intended to be limiting and include plural referents unless explicitly stated otherwise or the context clearly indicates otherwise.

The term “about” as used herein in reference to a number is used herein to include numbers which are greater, or less than, a stated or implied value by 1%, 5%, 10%, or 20%.

The terms “includes,” “comprises,” “including,” “comprising,” “has,” “having,” and grammatical variations thereof, when used in this specification, are not intended to be limiting, and specify the presence of stated features, elements, and/or components, but do not preclude the presence or addition of one or more other features, elements, components, and/or groups thereof.

Particular combinations of features are recited in the claims and/or disclosed in the specification, and these combinations of features are not intended to limit the disclosure of various aspects. Combinations of such features not specifically recited in the claims and/or disclosed in the specification. Although each dependent claim listed below may directly depend on only one claim, the disclosure of various aspects includes each dependent claim in combination with every other claim in the claim set. As used herein, a phrase referring to “at least one of” a list of items refers to any combination of those items, including single members. As an example, “at least one of: a, b, or c” is intended to cover a alone; b alone; c alone, a and b, a, b, and c, b and c, a and c, as well as any combination with multiples of the same element, such as a and a; a, a, and a; a, a, and b; a, a, and c; a, b, and b; a, c, and c; and any other combination or ordering of a, b, and c).

According to aspects of the present disclosure, a thermostable DNA polymerase of SEQ ID NO:1, and variants thereof, are provided.

(SEQ ID NO: 1)
MEAMLPLFEPKGRVLLVDGHHLAYRTFFALKGLTTSRGEPVQAVYGFAKS
LLKALKEDGYKAVFVVFDAKAPSFRHEAYEAYKAGRAPTPEDFPRQLALI
KELVDLLGFTRLEVPGYEADDVLATLAKKAEKEGYEVRILTADRDLYQLV
SDRVAVLHPEGHLITPEWLWEKYGLRPEQWVDFRALVGDPSDNLPGVKGI
GEKTALKLLKEWGSLENLLKNLDRVKPENVREKIKAHLEDLRLSLELSRV
RTDLPLEVDLAQGREPDREGLRAFLERLEFGSLLHEFGLLEAPAPLEEAP
WPPPEGAFVGFVLSRPEPMWAELKALAACRDGRVHRAADPLAGLKDLKEV
RGLLAKDLAVLASREGLDLVPGDDPMLLAYLLDPSNTTPEGVARRYGGEW
TEDAAHRALLSERLHRNLLKRLEGEEKLLWLYHEVEKPLSRVLAHMEATG
VRLDVAYLQALSLELAEEIRRLEEEVFRLAGHPFNLNSRDQLERVLFDEL
RLPALGKTQKTGKRSTSAAVLEALREAHPIVEKILQHRELTKLKNTYVDP
LPSLVHPRTGRLHTRFNQTATATGRLSSSDPNLQNIPVRTPLGQRIRRAF
VAEAGWALVALDYSQIELRVLAHLSGDENLIRVFQEGKDIHTQTASWMFG
VPPEAVDPLMRRAAKTVNFGVLYGMSAHRLSQELAIPYEEAVAFIERYFQ
SFPKVRAWIEKTLEEGRKRGYVETLFGRRRYVPDLNARVKSVREAAERMA
FNMPVQGTAADLMKLAMVKLFPRLREMGARMLLQVHDELLLEAPQARAEE
VAALAKEAMEKAYPLAVPLEVEVGMGEDWLSAKG

The term “thermostable” refers to a DNA polymerase enzyme that is stable and capable of performing a polymerization reaction at reaction temperatures, such as in the range of 40° C.-80° C. The term “thermostable” further refers to a DNA polymerase enzyme that is stable and retains polymerase activity upon repeated exposure to elevated temperatures, such as in the range of 80° C.-97° C.

The term “DNA polymerase” as used herein refers to an enzyme that catalyzes the polymerization of nucleotides, and encompasses enzymes that have activity to generate DNA from a DNA template and/or to generate DNA from an RNA template.

The term “RNA template” as used herein refers to RNA to be amplified using compositions and methods according to aspects of the present disclosure. An RNA template may be present in a test sample.

According to aspects of the present disclosure, the test sample is, or is derived from, a biological sample obtained from a mammalian subject. A biological sample obtained from a subject can be, but is not limited to, a sample of saliva, blood, plasma, serum, mucous, urine, feces, nasal material, cerebrospinal fluid, cerebroventricular fluid, pleural fluids, pulmonary and bronchial lavage samples, sweat, tears, semen, bladder wash samples, amniotic fluid, lymph, hair, skin, tumor, and peritoneal fluid.

A subject from which a sample is obtained can be any type of organism including, but not limited to, a mammal such as a human; a non-human primate; a rodent such as a mouse, rat, or guinea pig; a domesticated pet such as a cat or dog; a horse, cow, pig, sheep, goat, or rabbit; a bird, a reptile, an amphibian, an insect, bacteria, protozoa, a plant, or a virus. Subjects can be either gender and can be any age. In aspects of methods of the present disclosure, the subject is human.

According to aspects of the present disclosure, the test sample is, or is derived from, an environmental sample. An environmental sample can be, but is not limited to, a liquid, gas, or solid sample, including, but not limited to, a water sample, a sewage sample, an air sample, a surface swab, a food sample, a beverage sample, a clothing sample, and a soil sample. According to aspects of the present disclosure, the test sample is a sewage sample.

A test sample may be purified to remove or reduce undesirable contaminants, such as Mg²⁺, or other ions such as Fe²⁺, Fe³⁺, Hg⁺, Cd²⁺, Co²⁺, Cu²⁺, Ni²⁺, Zn²⁺, and Pb²⁺. A test sample may be purified to enrich for the RNA template. Purification to remove or reduce undesirable contaminants and/or to enrich for the RNA template is achieved using various well-known methods, such as, but not limited to, dilution, affinity chromatography, and size exclusion chromatography.

The term “variant” refers to a protein functional as a thermostable DNA polymerase and which includes an alteration, i.e. a substitution, insertion or deletion, of one, two, three, four, five, six, seven, or eight, amino acids compared to the full-length amino acid sequence of SEQ ID NO:1 while retaining a length in the range of 830 to 840 amino acids, such as having a length of 831 amino acids to 835 amino acids, such as having a length of 834 amino acids. According to aspects of the present disclosure, the term “variant” is used with the proviso that amino acid 453 of SEQ ID NO:1 is leucine. The term “variant” refers to proteins which have at least 99%, or greater, amino acid sequence identity to a full length sequence of SEQ ID NO:1, wherein the variant retains activity as a thermostable DNA polymerase.

Such additional amino acids can be added to a given amino acid sequence by any of various methods including, but not limited to, recombinant and synthetic DNA techniques, chemical conjugation and photoconjugation techniques.

A thermostable DNA polymerase of SEQ ID NO:1 or variant thereof according to aspects of the present disclosure can be generated by recombinant or synthetic DNA techniques in vitro, ex vivo, or in vivo, according to aspects of the present disclosure.

As will be readily apparent to one of skill in the art, due to the redundancy of the genetic code, more than one nucleic acid encodes a DNA polymerase of SEQ ID NO:1.

The term “nucleic acid” as used herein refers to RNA or DNA molecules having more than one nucleotide in any form including single-stranded, double-stranded, oligonucleotide or polynucleotide. The terms “nucleotide sequence” is used to refer to the ordering of nucleotides in an oligonucleotide or polynucleotide in a single-stranded form of nucleic acid.

The term “complementary” as used herein refers to Watson-Crick base pairing between nucleotides and specifically refers to nucleotides hydrogen bonded to one another with thymine or uracil residues linked to adenine residues by two hydrogen bonds and cytosine and guanine residues linked by three hydrogen bonds. In general, a nucleic acid includes a nucleotide sequence described as having a “percent complementarity” to a specified second nucleotide sequence. For example, a nucleotide sequence may have 80%, 90%, or 100% complementarity to a specified second nucleotide sequence, indicating that 8 of 10, 9 of 10 or 10 of 10 nucleotides of a sequence are complementary to the specified second nucleotide sequence. For instance, the nucleotide sequence 3′-TCGA-5′ is 100% complementary to the nucleotide sequence 5′-AGCT-3′. Further, the nucleotide sequence 3′-TCGA- is 100% complementary to a region of the nucleotide sequence 5′-TTAGCTGG-3′.

The terms “hybridization” and “hybridizes” refer to pairing and binding of complementary nucleic acids. Hybridization occurs to varying extents between two nucleic acids depending on factors such as the degree of complementarity of the nucleic acids, the melting temperature, Tm, of the nucleic acids and the stringency of hybridization conditions, as is well known in the art. The term “stringency of hybridization conditions” refers to conditions of temperature, ionic strength, and composition of a hybridization medium with respect to particular common additives such as formamide and Denhardt's solution. Determination of particular hybridization conditions relating to a specified nucleic acid is routine and is well known in the art, for instance, as described in J. Sambrook and D. W. Russell, Molecular Cloning: A Laboratory Manual, Cold Spring Harbor Laboratory Press; 3rd Ed., 2001; and F. M. Ausubel, Ed., Short Protocols in Molecular Biology, Current Protocols; 5th Ed., 2002. High stringency hybridization conditions are those which only allow hybridization of substantially complementary nucleic acids.

Typically, nucleic acids having about 85-100% complementarity are considered highly complementary and hybridize under high stringency conditions. Intermediate stringency conditions are exemplified by conditions under which nucleic acids having intermediate complementarity, about 50-84% complementarity, as well as those having a high degree of complementarity, hybridize. In contrast, low stringency hybridization conditions are those in which nucleic acids having a low degree of complementarity hybridize.

The terms “specific hybridization” and “specifically hybridizes” refer to hybridization of a particular nucleic acid to a target nucleic acid without substantial hybridization to nucleic acids other than the target nucleic acid in a sample.

Stringency of hybridization and washing conditions depends on several factors, including the Tm of the probe and target and ionic strength of the hybridization and wash conditions, as is well-known to the skilled artisan. Hybridization and conditions to achieve a desired hybridization stringency are described, for example, in Sambrook et al., Molecular Cloning: A Laboratory Manual, Cold Spring Harbor Laboratory Press, 2001; and Ausubel, F. et al., (Eds.), Short Protocols in Molecular Biology, Wiley, 2002.

An example of high stringency hybridization conditions is hybridization of nucleic acids over about 100 nucleotides in length in a solution containing 6X SSC, 5X Denhardt's solution, 30% formamide, and 100 micrograms/ml denatured salmon sperm at 37° C. overnight followed by washing in a solution of 0.1X SSC and 0.1% SDS at 60° C. for 15 minutes. SSC is 0.15M NaCl/0.015 M Na citrate. Denhardt's solution is 0.02% bovine serum albumin/0.02% FICOLL/0.02% polyvinylpyrrolidone.

Mutations can be introduced using standard molecular biology techniques, such as site-directed mutagenesis, PCR-mediated mutagenesis and chemical synthesis. One of skill in the art will recognize that one or more amino acid mutations can be introduced without altering the functional properties of the thermostable DNA polymerase of SEQ ID NO:1. For example, one or more amino acid substitutions, additions, or deletions can be made without altering the functional properties of the thermostable DNA polymerase of SEQ ID NO:1.

Conservative amino acid substitutions can be made in a thermostable DNA polymerase of SEQ ID NO:1, to produce variants according to aspects of the present disclosure. Conservative amino acid substitutions are art recognized substitutions of one amino acid for another amino acid having similar characteristics. For example, each amino acid may be described as having one or more of the following characteristics: electropositive, electronegative, aliphatic, aromatic, polar, hydrophobic and hydrophilic. A conservative substitution is a substitution of one amino acid having a specified structural or functional characteristic for another amino acid having the same characteristic. Acidic amino acids include aspartate, glutamate; basic amino acids include histidine, lysine, arginine; aliphatic amino acids include isoleucine, leucine and valine; aromatic amino acids include phenylalanine, histidine, tyrosine and tryptophan; polar amino acids include aspartate, glutamate, histidine, lysine, asparagine, glutamine, arginine, serine, threonine and tyrosine; and hydrophobic amino acids include alanine, cysteine, phenylalanine, glycine, isoleucine, leucine, methionine, proline, valine and tryptophan; and conservative substitutions include substitution among amino acids within each group. Amino acids may also be described in terms of relative size, alanine, cysteine, aspartate, glycine, asparagine, proline, threonine, serine, valine, all typically considered to be small.

Variants according to aspects of the present disclosure can include synthetic amino acid analogs, amino acid derivatives and/or non-standard amino acids, illustratively including, without limitation, alpha-aminobutyric acid, citrulline, canavanine, cyanoalanine, diaminobutyric acid, diaminopimelic acid, dihydroxy-phenylalanine, djenkolic acid, homoarginine, hydroxyproline, norleucine, norvaline, 3-phosphoserine, homoserine, 5-hydroxytryptophan, 1-methylhistidine, 3-methylhistidine, and ornithine.

To determine the percent identity of two amino acid sequences or of two nucleotide sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions X100%). In one embodiment, the two sequences are the same length. Alternatively, the two sequences may be different lengths, such as 1, 2, 3, 4, 5, 6, 7, 8, 9 or 10 amino acids different in length. The additions or deletions may be at the N-terminus, C-terminus, internally or a mixture of any thereof.

The determination of percent identity between two sequences can also be accomplished using a mathematical algorithm. A preferred, non-limiting example of a mathematical algorithm utilized for the comparison of two sequences is the algorithm of Karlin and Altschul, 1990, PNAS 87:2264 2268, modified as in Karlin and Altschul, 1993, PNAS. 90:5873 5877. Such an algorithm is incorporated into the NBLAST and XBLAST programs of Altschul et al., 1990, J. Mol. Biol. 215:403. BLAST nucleotide searches are performed with the NBLAST nucleotide program parameters set, e.g., for score=100, wordlength=12 to obtain nucleotide sequences homologous to a nucleic acid molecules of the present disclosure. BLAST protein searches are performed with the XBLAST program parameters set, e.g., to score 50, wordlength=3 to obtain amino acid sequences homologous to a protein molecule of the present disclosure. To obtain gapped alignments for comparison purposes, Gapped BLAST are utilized as described in Altschul et al., 1997, Nucleic Acids Res. 25:3389 3402. Alternatively, PSI BLAST is used to perform an iterated search which detects distant relationships between molecules. When utilizing BLAST, Gapped BLAST, and PSI Blast programs, the default parameters of the respective programs (e.g., of XBLAST and NBLAST) are used. Another preferred, non-limiting example of a mathematical algorithm utilized for the comparison of sequences is the algorithm of Myers and Miller, 1988, CABIOS 4:11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 is used.

The percent identity between two sequences is determined using techniques similar to those described above, with or without allowing gaps. In calculating percent identity, typically only exact matches are counted.

A thermostable DNA polymerase and variants thereof according to aspects of the present disclosure can be produced in recombinant host cells using well-known conventional techniques.

Broadly described, an expression cassette includes a nucleic acid encoding a thermostable DNA polymerase or variant thereof operably linked to one or more regulatory elements that control transcriptional expression of the nucleic acid. An expression cassette can be introduced into a host cell where it is expressed. The expression cassette can be included in an expression vector. The host cell can be in vitro, ex vivo, or in vivo. In the case where the host cell is in vitro or ex vivo, the thermostable DNA polymerase or variant thereof can be isolated from the host cell and administered to the subject in need thereof.

According to aspects of the present disclosure, the expression cassette includes a nucleic acid encoding a thermostable DNA polymerase of SEQ ID NO:1 or a variant thereof.

The term “regulatory element” as used herein refers to a nucleotide sequence which controls some aspect of the expression of an operably linked nucleic acid. Exemplary regulatory elements illustratively include an enhancer, an internal ribosome entry site (IRES), an intron; an origin of replication, a polyadenylation signal (pA), a promoter, a transcription termination sequence, and an upstream regulatory domain, which contribute to the replication, transcription, post-transcriptional processing of a nucleic acid. A secretory sequence encoding a secretion signal that directs an encoded heterologous protein into the secretory pathway of a host cell is optionally included. Additional sequences optionally included in an expression vector include one or more sequences encoding a marker suitable for selection of cells carrying the expression vector.

Those of ordinary skill in the art are capable of selecting and using these and other regulatory elements in an expression construct with no more than routine experimentation.

The term “operably linked” as used herein refers to a nucleic acid in functional relationship with a second nucleic acid. The term “operably linked” encompasses functional connection of two or more nucleic acids, such as an oligonucleotide or polynucleotide to be transcribed and a regulatory element such as a promoter or an enhancer element, which allows transcription of the nucleic acid to be transcribed.

The term “promoter” as used herein refers to a DNA sequence operably linked to a nucleic acid to be transcribed such as a nucleic acid encoding a desired molecule. A promoter is generally positioned upstream of a nucleic acid sequence to be transcribed and provides a site for specific binding by RNA polymerase and other transcription factors.

Particular promoters included in operable linkage with a nucleic acid molecule encoding a thermostable DNA polymerase or variant thereof are functional to preferentially express the operably-linked nucleic acid in a particular cell or tissue type. According to aspects of the present disclosure, an included promoter is a prokaryotic promoter, such as a bacterial promoter or bacterial phage promoter. According to aspects of the present disclosure, an included promoter is selected from promoters of high activity in E. coli such as the T7 phage promoter.

As will be recognized by the skilled artisan, the 5′ non-coding region of a gene can be isolated and used in its entirety as a promoter in an expression cassette to drive expression of an operably linked nucleic acid. Alternatively, a portion of the 5′ non-coding region can be isolated and inserted in an expression cassette to drive expression of an operably linked nucleic acid. In general, about 50-6000 bp of the 5′ non-coding region of a gene is included in an expression cassette to confer expression of the operably linked nucleic acid. Assays which are well-known in the art can be used to determine the ability of a designated portion of the 5′ non-coding region of a gene to drive expression of the operably linked nucleic acid.

Promoters described herein are known to be active in prokaryotic cells such as E. coli. Additional promoters useful in methods and compositions of the present disclosure may be determined to be active in a host cell system in culture or in vivo using conventional techniques, such as analysis of expression of RNA or protein produced from a nucleic acid expression construct in which the promoter is operably linked to a nucleic acid encoding the RNA or protein of the DNA polymerase. Promoter homologues and promoter variants can be included in an expression cassette according to the present disclosure. The terms “promoter homologue” and “promoter variant” refer to a promoter which has substantially similar functional properties to confer the desired type of expression on an operably linked nucleic acid compared to those disclosed herein.

The term “expression construct” is used herein to refer to a double-stranded recombinant DNA molecule containing a nucleic acid desired to be expressed and containing appropriate regulatory elements necessary or desirable for the transcription of the operably linked nucleic acid sequence in vitro or in vivo. The term “recombinant” is used to indicate a nucleic acid construct in which two or more nucleic acids are linked and which are not found linked in nature. The term “expressed” refers to transcription of a nucleic acid to produce a corresponding mRNA and/or translation of the mRNA to produce the corresponding protein. Expression constructs can be generated recombinantly or synthetically or by DNA synthesis using well-known methodology.

An expression construct is introduced into a cell using well-known methodology, such as, but not limited to, by introduction of a vector containing the expression construct into the cell. A “vector” is a nucleic acid that transfers an inserted nucleic acid into and/or between host cells becoming self-replicating. The term includes vectors that function primarily for insertion of a nucleic acid into a cell, replication of vectors that function primarily for the replication of nucleic acid, and expression vectors that function for transcription and/or translation of a nucleic acid. Also included are vectors that provide more than one of the above functions.

Expression vectors include plasmids, phages, viruses, BACs, YACs, and the like.

Prokaryotic expression vectors can be used to express a thermostable DNA polymerase or variant thereof of the present disclosure. Non-limiting examples of prokaryotic expression systems include plasmids, bacterial phages and eukaryotic viruses.

A host cell for expression of a thermostable DNA polymerase or variant thereof can be prokaryotic or eukaryotic, such as bacterial, plant, insect, fungus, yeast, and mammalian cells.

An expression vector is introduced into a host cell using well-known techniques such as transformation, infection or transfection, including use of competent bacterial cells, calcium phosphate transfection, liposome-mediated transfection, electroporation, sonoporation and nanoparticle-based methodologies. Expression constructs and methods for their generation and use to express a desired protein are known in the art, as described, for example, in Sambrook et al., Molecular Cloning: A Laboratory Manual, Cold Spring Harbor Laboratory Press, 2001; Ausubel, F. et al., (Eds.), Short Protocols in Molecular Biology, Wiley, 2002; and S. J. Higgins and B. D. Hames (Eds.), Protein Expression: A Practical Approach, Oxford University Press, USA, 1999.

In addition to recombinant methodology, chemical synthetic techniques can be used to produce a desired thermostable DNA polymerase or variant thereof. For example, a thermostable DNA polymerase or variant thereof can be produced using solid phase synthesis, solution phase synthesis, partial solid phase synthesis or fragment condensation.

The term “isolated” as used herein refers to a substance that has been separated from contaminating materials. Generally, an isolated substance described herein is at least about 80% pure, at least about 90% pure, at least about 95% pure, or greater than about 99% pure. Purification is achieved using well-known standard methodology such as fractionation and/or chromatography, such as heat precipitation, isoelectric point precipitation, ammonium sulfate precipitation and elution chromatography such as size exclusion chromatography, displacement chromatography, ion exchange chromatography and bioaffinity chromatography. Exemplary purification methodology is described in S. Doonan, Protein Purification Protocols Humana Press, 1996.

Compositions are provided according to aspects of the present disclosure which include: a recombinant thermostable DNA polymerase comprising SEQ ID NO:1 or a variant thereof having at least 99% identity to SEQ ID NO:1; and a reaction buffer, with the proviso that the reaction buffer does not include Mg²⁺ in a significant amount.

According to aspects of the present disclosure, the reaction buffer contains no more than 0.1 mM Mg²⁺.

According to aspects of the present disclosure, the reaction buffer contains a Tris (Tris(hydroxymethyl)aminomethane) buffer. According to aspects of the present disclosure, the reaction buffer contains a Tris-HCl buffer.

According to aspects of the present disclosure, the reaction buffer contains 10-30 mM Tris-HCl, pH 8.5-9.0.

According to aspects of the present disclosure, the reaction buffer contains 10, 11, 12, 13, 14, 15, 16, 17, 18, 19, 20, 21, 22, 23, 24, 25, 26, 27, 28, 29, or 30 mM Tris-HCl, pH 8.5-9.0.

According to aspects of the present disclosure, the reaction buffer contains a potassium salt. According to aspects of the present disclosure, the reaction buffer contains KCl.

According to aspects of the present disclosure, the reaction buffer contains 20-40 mM KCl.

According to aspects of the present disclosure, the reaction buffer contains 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, or 40 mM KCl.

According to aspects of the present disclosure, the reaction buffer contains 30 mM KCl.

According to aspects of the present disclosure, the reaction buffer contains Mn²⁺.

According to aspects of the present disclosure, the reaction buffer contains 1.5-3.5 mM Mn²⁺. Any source of Mn²⁺ compatible with a PCR reaction may be used.

According to aspects of the present disclosure, the reaction buffer contains 1.5 mM Mn²⁺, 2.0 mM Mn²⁺, 2.5 mM Mn²⁺, 3.0 mM Mn²⁺, or 3.5 mM Mn²⁺.

According to aspects of the present disclosure, the reaction buffer contains 1.5 mM Mn²⁺, 1.6 mM Mn²⁺, 1.7 mM Mn²⁺, 1.8 mM Mn²⁺, 1.9 mM Mn²⁺, 2.0 mM Mn²⁺, 2.1 mM Mn²⁺, 2.2 mM Mn²⁺, 2.3 mM Mn²⁺, 2.4 mM Mn²⁺, 2.5 mM Mn²⁺, 2.6 mM Mn²⁺, 2.7 mM Mn²⁺, 2.8 mM Mn²⁺, 2.9 mM Mn²⁺, 3.0 mM Mn²⁺, 3.1 mM Mn²⁺, 3.2 mM Mn²⁺, 3.3 mM Mn²⁺, 3.4 mM Mn²⁺, or 3.5 mM Mn²⁺.

According to aspects of the present disclosure, the reaction buffer contains 1.5-3.5 mM manganese chloride, manganese acetate, manganese gluconate, manganese sulfate, or a combination of any two or more thereof, such that the reaction buffer contains 1.5 mM Mn²⁺, 1.6 mM Mn²⁺, 1.7 mM Mn²⁺, 1.8 mM Mn²⁺, 1.9 mM Mn²⁺, 2.0 mM Mn²⁺, 2.1 mM Mn²⁺, 2.2 mM Mn²⁺, 2.3 mM Mn²⁺, 2.4 mM Mn²⁺, 2.5 mM Mn²⁺, 2.6 mM Mn²⁺, 2.7 mM Mn²⁺, 2.8 mM Mn²⁺, 2.9 mM Mn²⁺, 3.0 mM Mn²⁺, 3.1 mM Mn²⁺, 3.2 mM Mn²⁺, 3.3 mM Mn²⁺, 3.4 mM Mn²⁺, or 3.5 mM Mn²⁺.

According to aspects of the present disclosure, the reaction buffer contains 1.5 mM manganese chloride, 1.6 mM manganese chloride, 1.7 mM manganese chloride, 1.8 mM manganese chloride, 1.9 mM manganese chloride, 2.0 mM manganese chloride, 2.1 mM manganese chloride, 2.2 mM manganese chloride, 2.3 mM manganese chloride, 2.4 mM manganese chloride, 2.5 mM manganese chloride, 2.6 mM manganese chloride, 2.7 mM manganese chloride, 2.8 mM manganese chloride, 2.9 mM manganese chloride, 3.0 mM manganese chloride, 3.1 mM manganese chloride, 3.2 mM manganese chloride, 3.3 mM manganese chloride, 3.4 mM manganese chloride, or 3.5 mM manganese chloride.

According to aspects of the present disclosure, the reaction buffer contains 1.5 mM manganese sulfate, 1.6 mM manganese sulfate, 1.7 mM manganese sulfate, 1.8 mM manganese sulfate, 1.9 mM manganese sulfate, 2.0 mM manganese sulfate, 2.1 mM manganese sulfate, 2.2 mM manganese sulfate, 2.3 mM manganese sulfate, 2.4 mM manganese sulfate, 2.5 mM manganese sulfate, 2.6 mM manganese sulfate, 2.7 mM manganese sulfate, 2.8 mM manganese sulfate, 2.9 mM manganese sulfate, 3.0 mM manganese sulfate, 3.1 mM manganese sulfate, 3.2 mM manganese sulfate, 3.3 mM manganese sulfate, 3.4 mM manganese sulfate, or 3.5 mM manganese sulfate.

According to aspects of the present disclosure, the reaction buffer contains 1.5 mM manganese acetate, 1.6 mM manganese acetate, 1.7 mM manganese acetate, 1.8 mM manganese acetate, 1.9 mM manganese acetate, 2.0 mM manganese acetate, 2.1 mM manganese acetate, 2.2 mM manganese acetate, 2.3 mM manganese acetate, 2.4 mM manganese acetate, 2.5 mM manganese acetate, 2.6 mM manganese acetate, 2.7 mM manganese acetate, 2.8 mM manganese acetate, 2.9 mM manganese acetate, 3.0 mM manganese acetate, 3.1 mM manganese acetate, 3.2 mM manganese acetate, 3.3 mM manganese acetate, 3.4 mM manganese acetate, or 3.5 mM manganese acetate.

According to aspects of the present disclosure, the reaction buffer contains 1.5 mM manganese gluconate, 1.6 mM manganese gluconate, 1.7 mM manganese gluconate, 1.8 mM manganese gluconate, 1.9 mM manganese gluconate, 2.0 mM manganese gluconate, 2.1 mM manganese gluconate, 2.2 mM manganese gluconate, 2.3 mM manganese gluconate, 2.4 mM manganese gluconate, 2.5 mM manganese gluconate, 2.6 mM manganese gluconate, 2.7 mM manganese gluconate, 2.8 mM manganese gluconate, 2.9 mM manganese gluconate, 3.0 mM manganese gluconate, 3.1 mM manganese gluconate, 3.2 mM manganese gluconate, 3.3 mM manganese gluconate, 3.4 mM manganese gluconate, or 3.5 mM manganese gluconate. According to aspects of the present disclosure, the reaction buffer contains (NH₄)₂SO₄.

According to aspects of the present disclosure, the reaction buffer contains 5-15 mM (NH₄)₂SO₄. According to aspects of the present disclosure, the reaction buffer contains 5 mM, (NH₄)₂SO₄, 6 mM (NH₄)₂SO₄, 7 mM (NH₄)₂SO₄, 8 mM (NH₄)₂SO₄, 9 mM (NH₄)₂SO₄, 10 mM (NH₄)₂SO₄, 11 mM (NH₄)₂SO₄, 12 mM, (NH₄)₂SO₄, 13 mM (NH₄)₂SO₄, 14 mM (NH₄)₂SO₄, or 15 mM (NH₄)₂SO₄. According to aspects of the present disclosure, the reaction buffer contains 10 mM (NH₄)₂SO₄.

According to aspects of the present disclosure, the reaction buffer contains gelatin and/or serum albumin. According to aspects of the present disclosure, the reaction buffer contains 0.01-0.20% (w/v) gelatin and/or serum albumin. According to aspects of the present disclosure, the reaction buffer contains 0.01%, 0.02%, 0.03%, 0.04%, 0.05%, 0.06%, 0.07%, 0.08%, 0.09%, 0.10%, 0.11%, 0.12%, 0.13%, 0.14%, 0.15%, 0.16%, 0.17%, 0.18%, 0.19%, or 0.20% (w/v) gelatin and/or serum albumin. According to aspects of the present disclosure, the reaction buffer contains 0.10% (w/v) gelatin and/or serum albumin.

According to aspects of the present disclosure, the reaction buffer contains a detergent. According to aspects of the present disclosure, the reaction buffer contains a nonionic surfactant. According to aspects of the present disclosure, the reaction buffer contains polysorbate 20.

According to aspects of the present disclosure, the reaction buffer contains 0.05%, 0.06%, 0.07%, 0.08%, 0.09%, 0.10%, 0.11%, 0.12%, 0.13%, 0.14%, or 0.15% (w/v) of a detergent.

According to aspects of the present disclosure, the reaction buffer contains 0.05%, 0.06%, 0.07%, 0.08%, 0.09%, 0.10%, 0.11%, 0.12%, 0.13%, 0.14%, or 0.15% (w/v) of a nonionic detergent.

According to aspects of the present disclosure, the reaction buffer contains 0.05%, 0.06%, 0.07%, 0.08%, 0.09%, 0.10%, 0.11%, 0.12%, 0.13%, 0.14%, or 0.15% (w/v) of polysorbate 20.

According to aspects of the present disclosure, the reaction buffer contains 10-30 mM Tris-HCl, pH 8.5-9.0; 20-40 mM KCl; 5-15 mM (NH₄)₂SO₄; 1.5-3.5 mM Mn²⁺; 0.01-0.20% (w/v) gelatin and/or serum albumin; and 0.05-0.15% (w/v) of a nonionic detergent, with the proviso that no more than 0.1 mM Mg²⁺ is present in the composition.

According to aspects of the present disclosure, the reaction buffer contains 10-30 mM Tris-HCl, pH 8.5-9.0; 20-40 mM KCl; 5-15 mM (NH₄)₂SO₄; 1.5-3.5 mM manganese chloride, manganese acetate, manganese gluconate, manganese sulfate, or any two or more thereof; 0.01-0.20% (w/v) gelatin and/or serum albumin; and 0.05-0.15% (w/v) of a nonionic detergent, with the proviso that no more than 0.1 mM Mg²⁺ is present in the composition.

According to aspects of the present disclosure, the reaction buffer contains 25 mM Tris-HCl, pH 8.8; 30 mM KCl; 10 mM (NH₄)₂SO₄; 2.0 mM or 2.5 mM MnCl₂; 0.1% (w/v) gelatin and/or serum albumin; and 0.1% (w/v) of a nonionic detergent, with the proviso that no more than 0.1 mM Mg²⁺ is present in the composition.

According to aspects of the present disclosure, the reaction buffer contains a chelating agent effective to bind and sequester divalent or trivalent metal ions including, but not limited to, Mg²⁺, Co²⁺, Ni²⁺, Cu²⁺, Zn²⁺, Hg₂²⁺, Fe²⁺, Cd²⁺, Pb²⁺, and Ca²⁺, wherein the chelating agent has a lower binding affinity for Mn²⁺ than for Fe²⁺, Fe³⁺, Hg⁺, Cd²⁺, Co²⁺, Cu²⁺, Ni²⁺, Zn²⁺, and Pb²⁺.

According to aspects of the present disclosure, the reaction buffer contains a chelating agent which has a lower binding affinity for Mn²⁺ than for Fe²⁺, Fe³⁺, Hg⁺, Cd²⁺, Co²⁺, Cu²⁺, Ni²⁺, Zn²⁺, and Pb²⁺, including, but not limited to, N-(2-hydroxyethyl)ethylenediaminetriacetic acid (HEDTA), ethylenediaminetetraacetic acid (EDTA), ethyleneglycol bis(2-aminoethyl ether)-N,N,N′,N′ tetraacetic acid (EGTA), diethylenetriaminepentaacetic acid (DTPA), trans-1,2-diaminocyclohexane-N,N,N′,N′-tetraacetic acid (CDTA), nitrilotriacetic acid (NTA), and a combination of any two or more thereof.

According to aspects of the present disclosure, the chelating agent has a concentration in the reaction mixture in the range of 0.01 mM-0.1 mM. According to aspects of the present disclosure, a chelating agent has a concentration in the reaction mixture in the range of 0.01 mM, 0.02 mM, 0.03 mM, 0.04 mM, 0.05 mM, 0.06 mM, 0.07 mM, 0.08 mM, 0.09 mM, or 0.1 mM.

According to aspects of the present disclosure, the reaction buffer contains 10-30 mM Tris-HCl, pH 8.5-9.0; 20-40 mM KCl; 5-15 mM (NH₄)₂SO₄; 1.5-3.5 mM Mn²⁺; 0.01-0.20% (w/v) gelatin and/or serum albumin; 0.05-0.15% (w/v) of a nonionic detergent, and 0.01 mM-0.1 mM HEDTA, EDTA, EGTA, DTPA, CDTA, NTA, or any two or more thereof, with the proviso that no more than 0.1 mM Mg²⁺ is present in the composition.

According to aspects of the present disclosure, the reaction buffer contains 10-30 mM Tris-HCl, pH 8.5-9.0; 20-40 mM KCl; 5-15 mM (NH₄)₂SO₄; 1.5-3.5 mM manganese chloride, manganese acetate, manganese gluconate, manganese sulfate, or any two or more thereof; 0.01-0.20% (w/v) gelatin and/or serum albumin; 0.05-0.15% (w/v) of a nonionic detergent, and 0.01 mM-0.1 mM HEDTA, EDTA, EGTA, DTPA, CDTA, NTA, or any two or more thereof, with the proviso that no more than 0.1 mM Mg²⁺ is present in the composition.

According to aspects of the present disclosure, the reaction buffer contains 25 mM Tris-HCl, pH 8.8; 30 mM KCl; 10 mM (NH₄)₂SO₄; 1.5-3.5 mM Mn²⁺; 0.1% (w/v) gelatin and/or serum albumin; 0.1% (w/v) of a nonionic detergent, and 0.01 mM-0.1 mM HEDTA, EDTA, EGTA, DTPA, CDTA, NTA, or any two or more thereof, with the proviso that no more than 0.1 mM Mg²⁺ is present in the composition.

cording to aspects of the present disclosure, the reaction buffer contains 0.01 mM-0.1 mM HEDTA, with the proviso that no more than 0.1 mM Mg²⁺ is present in the composition.

Nucleotides, including, but not limited to, deoxynucleotide triphosphates (dNTPs) and analogs thereof, labeled or unlabeled, can be included reaction mixtures according to methods of the present disclosure.

According to aspects of the present disclosure, the reaction buffer contains dNTPs. The term “dNTP” refers deoxyribonucleoside triphosphates, dATP, dCTP, dGTP, dTTP, and dUTP.

The term “nucleotide analog” in this context refers to a modified or non-naturally occurring nucleotide which can be polymerized with nucleotides and/or nucleotide analogs by template directed nucleic acid amplification catalyzed by a DNA polymerase.

Particular nucleotide analogs are capable of Watson-Crick pairing via hydrogen bonds with a complementary nucleotide and illustratively include, but are not limited to, those containing an analog of a nucleotide base such as substituted purines or pyrimidines, deazapurines, methylpurines, methylpyrimidines, aminopurines, aminopyrimidines, thiopurines, thiopyrimidines, indoles, pyrroles, 7-deazaguanine, 7-deazaadenine, 7-methylguanine, hypoxanthine, pseudocytosine, pseudoisocytosine, isocytosine, isoguanine, 2-thiopyrimidines, 4-thiothymine, 6-thioguanine, nitropyrrole, nitroindole, and 4-methylindole.

Nucleotide analogs include those containing an analog of a deoxyribose such as a substituted deoxyribose, a substituted or non-substituted arabinose, a substituted or non-substituted xylose, and a substituted or non-substituted pyranose.

Nucleotide analogs include those containing an analog of a phosphate ester such as phosphorothioates, phosphorodithioates, phosphoroamidates, phosphoroselenoates, phosophoroanilothioates, phosphoroanilidates, phosphoroamidates, boronophosphates, phosphotriesters, and alkylphosphonates such as methylphosphonates.

According to aspects of the present disclosure, the reaction buffer contains 20-200 μM nucleotides and/or nucleotide analogs. According to aspects of the present disclosure, the reaction buffer contains 50-400 μM nucleotides and/or nucleotide analogs.

According to aspects of the present disclosure the composition containing the thermostable DNA polymerase and the reaction buffer is liquid, frozen, or lyophilized.

According to aspects of the present disclosure the composition containing the thermostable DNA polymerase and the reaction buffer is stored at a temperature in the range of −80° C. to 30° C.

Methods of amplifying an RNA template are provided according to aspects of the present disclosure, which include: providing a composition including a thermostable DNA polymerase of SEQ ID NO:1 or a variant thereof; adding an RNA template, a reverse transcription primer, and a pair of amplification primers, of which one can be identical to the reverse transcription primer, to the composition in a container, producing a reaction mixture in the container; incubating the reaction mixture in the container at a denaturing temperature in the range of 92° C. to 97° C. for 0.5 to 5 minutes, producing denatured RNA template in the reaction mixture in the container; incubating the reaction mixture in the container at a reverse transcription temperature in the range of 70° C. to 77° C. for 1 to 20 minutes, producing cDNA in the reaction mixture in the container; and amplifying the cDNA in the reaction mixture in the container by an amplification method.

The terms “amplification method” and “amplification reaction” refer to an in vitro method for copying a template target nucleic acid, thereby producing nucleic acids which include copies of all or a portion of the template target nucleic acid. Amplification methods used according to aspects of the present disclosure are those which include template directed primer extension catalyzed by a recombinant thermostable DNA polymerase including SEQ ID NO:1, or a variant thereof having at least 99% identity to SEQ ID NO:1 using a pair of primers which flank the target nucleic acid, illustratively including, but not limited to, Polymerase Chain Reaction (PCR), and other nucleic acid amplification methods, for instance, as described in C.W. Dieffenbach et al., PCR Primer: A Laboratory Manual, Cold Spring Harbor Laboratory Press, 2003; and V. Demidov et al., DNA Amplification: Current Technologies and Applications, Taylor & Francis, 2004.

The term “primer” refers to an oligonucleotide that is capable of acting as a site of initiation of synthesis of a template directed primer extension product under appropriate reaction conditions. An oligonucleotide primer is typically about 10-30 contiguous nucleotides in length and may be longer or shorter. An oligonucleotide primer is completely or substantially complementary to a region of a template nucleic acid such that, under hybridization conditions, the oligonucleotide primer anneals to the complementary region of the template nucleic acid. Primer design for reverse transcription and/or amplification of a target nucleic acid is well-known to those of skill in the art. Primers for reverse transcription and/or amplification of a target nucleic acid are designed according to well-known methods and criteria. For instance, the annealing temperature of amplification primers should be about the same, within a few degrees; primers should not form dimers with each other; and primers should not form secondary structures, such as hairpins. Methods and considerations for primer design are known in the art, such as described in, for example, Yuryev, A., PCR Primer Design, Methods in Molecular Biology, vol. 42, Human Press, 2007; C. W. Dieffenbach et al., PCR Primer: A Laboratory Manual, Cold Spring Harbor Laboratory Press, 2003; and V. Demidov et al., DNA Amplification: Current Technologies and Applications, Taylor & Francis, 2004.

Methods according to aspects of the present disclosure can be performed in any suitable container. Examples of such containers include, but are not limited to, reaction vessels such as reaction tubes. A container can be a multi-chamber container. Multi-chamber containers illustratively include, but are not limited to, multi-well strips and plates, multi-depression substrates such as slides, silicon chips or trays.

Kits for reverse transcription and amplification are provided according to aspects of the present disclosure which include a DNA polymerase of SEQ ID NO:1, or a variant thereof, disposed in a reaction buffer in a reaction container. An ancillary reagent may be included in a kit of the present disclosure, such as one or more primers, target nucleotide control or standards, one or more additional reaction containers, an additional buffer, a rehydrating solution, a detectable label, a detection reagent, and the like.

Embodiments of inventive compositions and methods are illustrated in the following examples. These examples are provided for illustrative purposes and are not considered limitations on the scope of inventive compositions and methods.

### EXAMPLES

In this example, a reaction mixture containing all reagents for reverse transcription of an RNA template and PCR reagents for subsequent amplification of the cDNA was made, including the DNA polymerase of SEQ ID NO:1. The reaction mix contained 100 nM each of the forward and reverse primers, 1 ng of the RNA template, 200 μM dNTPs, 10 mM KCl, 10 mM (NH₄)₂SO₄, 20 mM Tris-HCl pH 8.75, 2 mM MnCl₂, 0.1% Triton-X100, and 0.1% gelatin.

The reaction mixture was incubated under conditions for reverse transcription of the RNA template to produce cDNA.
94° C. 3 min
60-72° C.* 10 min (* adjustable based on primers used)

The following thermocycling protocol (amplification reaction) was used to amplify the cDNA for 25 μL reactions using a pair of amplification primers, of which one can be the reverse transcription primer:
94° C. 1 min
94° C. 15 sec −58° C.* 30 sec for 50 cycles or as appropriate for the particular cDNA
40° C. 30 sec

Agarose gel analysis was performed for quality control and validation of target specificity.

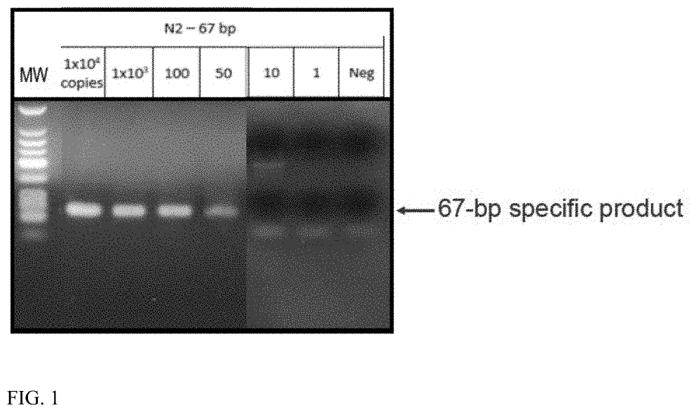

The agarose gel electrophoresis result shown in FIG. 1 shows the final yield of DNA amplified from various copies, 1−1×10⁴ of synthetic SARS-CoV-2 RNA standard.

The initial result demonstrated effective detection of SARS-CoV-2 RNA target of 50 or less copies.

### Example

DNA polymerase activity in PCR

25 μL PCR of serial dilutions of the modified DNA polymerase (SEQ ID NO:1) were run with a pair of primers flanking an ˜560 bp segment of cDNA encoding mouse cardiac troponin I. The reaction mix contains 100 nM each of the forward and reverse primers, 1 ng of the recombinant plasmid DNA, 200 μM dNTPs, 10 mM KCl, 10 mM (NH₄)₂SO₄, 20 mM Tris-HCl pH 8.75, 2 mM MnCl₂, 0.1% Triton-X100, and 0.1% gelatin. The amplification reaction steps are: 94° C. for 2 minutes, 35 cycles of 94° C. 30 seconds—55° C. 30 seconds—72° C. 60 seconds, and a final extension at 72° C. for 5 minutes. A no-template negative control was included in all tests. 10 μL of the PCR products were analyzed on 1.5% agarose gel in Tris-acetate-EDTA buffer containing 10 μg/mL ethidium bromide. The resulting gel was photographed on a UV light box.

### Example

One tube single enzyme RT-PCR using the modified DNA polymerase

The modified DNA polymerase (SEQ ID NO:1) was used for one tube single enzyme RT-PCR amplifications. 20 μL reactions were done with standard SARS-CoV2 N1F/N1R primer pairs (5′-GACCCCAAAATCAGCGAAAT-3′ (SEQ ID NO:2) and 5′-TCTGGTTACTGCCAGTTGAATCTG-3′ (SEQ ID NO:3)) using the following steps: 72° C. 20 minutes for reverse transcription and first strand cDNA synthesis, 95° C. 3 minutes, followed by the amplification reaction: 40-50 PCR cycles of 95° C. 15 seconds −60° C. 30 seconds.

Reactions with SARS-CoV2 primer pairs N2F/N2R (5′-TTACAAACATTGGCCGCAAA-3′ (SEQ ID NO:4) and 5′-GCGCGACATTCCGAAGAA-3′ (SEQ ID NO:5)) were run similarly: 72° C. 20 minutes for reverse transcription and first strand cDNA synthesis, 95° C. 3 minutes, followed by the amplification reaction: 40-50 PCR cycles of 95° C. 15 seconds −58° C. 30 seconds.

All reactions included a no-template negative control. 10 μL of the PCR products were analyzed using 4% agarose gels containing 10 μg/mL ethidium bromide.

The RT-PCR mix contains the modified DNA polymerase (SEQ ID NO:1), 400 nM each of forward and reverse primers, 1 ng of the RNA template, 500 μM dNTPs, 40 mM KCl, 10 mM (NH₄)₂SO₄, 25 mM Tris-HCl pH 8.8, 2.0 mM MnCl₂, 0.1% Triton-X100, 0.1% gelatin, and 50 μM HEDTA (N-(2-hydroxyethyl)ethylenediaminetriacetic acid).

### Example

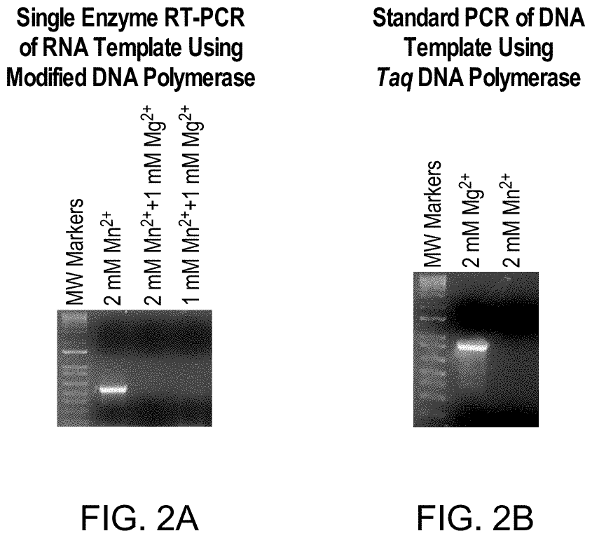

FIG. 2A is an agarose gel showing that the modified DNA polymerase (SEQ ID NO:1) gives effective amplification of an RNA target segment in the presence of 2 mM Mn²⁺ and the presence of Mg²⁺ diminished the polymerase activity. FIG. 2B is an agarose gel showing that in a control standard PCR using Taq polymerase and DNA template, the presence of Mg²⁺ but not Mn²⁺ gives the expected polymerase activity.

### Example

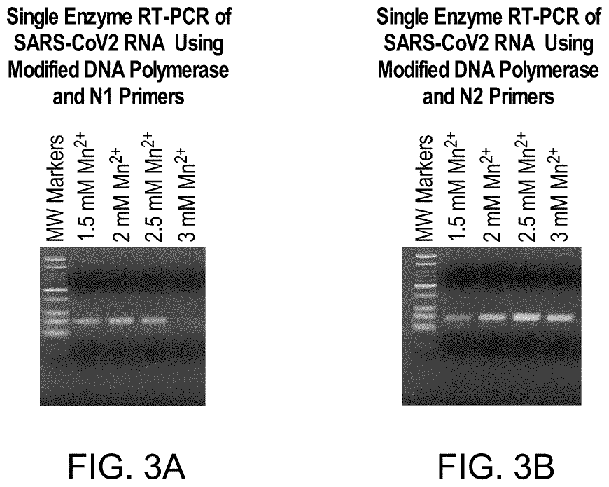

FIGS. 3A and 3B are images of agarose gels showing that various concentrations of Mn²⁺ may be needed for effective amplification using different primer pairs and/or targeting sequences for the amplifications of SARS-CoV2 RNA targets segment using the modified DNA polymerase (SEQ ID NO:1).

The RT-PCR reaction mixture contains the modified DNA polymerase (SEQ ID NO:1), 400 nM each of forward and reverse primers, 500 μM dNTPs, 40 mM KCl, 10 mM (NH₄)₂SO₄, 25 mM Tris-HCl pH 8.8, 0.1% Triton-X100, 0.1% gelatin and 50 μM HEDTA (N-(2-hydroxyethyl)ethylenediaminetriacetic acid). Mn²⁺ was included in the reaction mix in a concentration of 1.5 mM Mn²⁺, 2.0 mM Mn²⁺, 2.5 mM Mn²⁺, or 3.0 mM Mn²⁺.

FIG. 3A: N1 primers with 77-bp product. FIG. 3B: N2 primers with 74-bp product.

Any patents or publications mentioned in this specification are incorporated herein by reference to the same extent as if each individual publication is specifically and individually indicated to be incorporated by reference.

The compositions and methods described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

1. A composition, comprising: a recombinant thermostable DNA polymerase comprising SEQ ID NO:1 or a variant thereof having at least 99% identity to SEQ ID NO:1; and a reaction buffer, the reaction buffer comprising 10-30 mM Tris-HCl, pH 8.5-9.0; 20-40 mM KCl; 5-15 mM (NH₄)₂SO₄; 1.5-3.5 mM Mn²⁺; 0.01-0.20% (w/v) gelatin and/or serum albumin; and 0.05-0.15% (w/v) of a nonionic detergent, with the proviso that no more than 0.1 mM Mg²⁺ is present in the composition.

2. The composition of claim 1, wherein the reaction buffer further comprises a chelating agent.

3. The composition of claim 2, wherein the chelating agent is selected from the group consisting of: N-(2-hydroxyethyl)ethylenediaminetriacetic acid (HEDTA), ethylenediaminetetraacetic acid (EDTA), ethyleneglycol bis(2-aminoethyl ether)-N,N,N′,N′ tetraacetic acid (EGTA), diethylenetriaminepentaacetic acid (DTPA), trans-1,2-diaminocyclohexane-N,N,N′,N′-tetraacetic acid (CDTA), nitrilotriacetic acid (NTA), and a combination of any two or more thereof.

4. The composition of claim 2, wherein the chelating agent is HEDTA

5. The composition of claim 2, wherein the chelating agent is present in a concentration of 0.01-0.1 mM.

6. The composition of claim 1, wherein the reaction buffer comprising 25 mM Tris-HCl, pH 8.8; 30 mM KCl; 10 mM (NH₄)₂SO₄; 1.5-3.5 mM Mn²⁺; 0.1% (w/v) gelatin and/or serum albumin; and 0.1% (w/v) of a nonionic detergent.

7. The composition of claim 6, wherein the reaction buffer further comprises a chelating agent.

8. The composition of claim 7, wherein the chelating agent is selected from the group consisting of: N-(2-hydroxyethyl)ethylenediaminetriacetic acid (HEDTA), ethylenediaminetetraacetic acid (EDTA), ethyleneglycol bis(2-aminoethyl ether)-N,N,N′,N′ tetraacetic acid (EGTA), diethylenetriaminepentaacetic acid (DTPA), trans-1,2-diaminocyclohexane-N,N,N′,N′-tetraacetic acid (CDTA), nitrilotriacetic acid (NTA), and a combination of any two or more thereof.

9. The composition of claim 7, wherein the chelating agent is HEDTA.

10. The composition of claim 7, wherein the chelating agent is present in a concentration of 0.01-0.1 mM.

11. The composition of claim 1, wherein the nonionic detergent is polysorbate 20.

12. The composition of claim 1, further comprising dNTPs.

13. The composition of claim 1, wherein the composition is liquid, frozen or lyophilized.

14. The composition of claim 1, wherein the composition is stored at a temperature in the range of −80° C. to 30° C.

15. A method of amplifying an RNA template, comprising:
providing a composition according to claim 1;
adding an RNA template, a reverse transcription primer, and a pair of amplification primers, to the composition in a container, producing a reaction mixture in the container;
incubating the reaction mixture in the container at a denaturing temperature in the range of 92° C. to 97° C. for 0.5 to 5 minutes, producing denatured RNA template in the reaction mixture in the container;
incubating the reaction mixture in the container at a reverse transcription temperature in the range of 70° C. to 77° C. for 1 to 20 minutes, producing cDNA in the reaction mixture in the container; and
amplifying the cDNA in the reaction mixture in the container by an amplification reaction.

16. The method of claim 15, wherein one of the pair of amplification primers is identical to the reverse transcription primer.


SEQUENCE LISTING (5 sequences)

SEQ ID NO: 1 - protein, 834 aa, Thermus thermophilus
MEAMLPLFEPKGRVLLVDGHHLAYRTFFALKGLTTSRGEPVQAVYGFAKSLLKALKEDGYKAVFVVFDAKAPSFRHEAYEAYKAGRAPTPEDFPRQLALIKELVDLLGFTRLEVPGYEADDVLATLAKKAEKEGYEVRILTADRDLYQLVSDRVAVLHPEGHLITPEWLWEKYGLRPEQWVDFRALVGDPSDNLPGVKGIGEKTALKLLKEWGSLENLLKNLDRVKPENVREKIKAHLEDLRLSLELSRVRTDLPLEVDLAQGREPDREGLRAFLERLEFGSLLHEFGLLEAPAPLEEAPWPPPEGAFVGFVLSRPEPMWAELKALAACRDGRVHRAADPLAGLKDLKEVRGLLAKDLAVLASREGLDLVPGDDPMLLAYLLDPSNTTPEGVARRYGGEWTEDAAHRALLSERLHRNLLKRLEGEEKLLWLYHEVEKPLSRVLAHMEATGVRLDVAYLQALSLELAEEIRRLEEEVFRLAGHPFNLNSRDQLERVLFDELRLPALGKTQKTGKRSTSAAVLEALREAHPIVEKILQHRELTKLKNTYVDPLPSLVHPRTGRLHTRFNQTATATGRLSSSDPNLQNIPVRTPLGQRIRRAFVAEAGWALVALDYSQIELRVLAHLSGDENLIRVFQEGKDIHTQTASWMFGVPPEAVDPLMRRAAKTVNFGVLYGMSAHRLSQELAIPYEEAVAFIERYFQSFPKVRAWIEKTLEEGRKRGYVETLFGRRRYVPDLNARVKSVREAAERMAFNMPVQGTAADLMKLAMVKLFPRLREMGARMLLQVHDELLLEAPQARAEEVAALAKEAMEKAYPLAVPLEVEVGMGEDWLSAKG

SEQ ID NO: 2 - DNA, 20 bp, synthetic construct
  source 1..20: mol_type = other DNA
gaccccaaaatcagcgaaat

SEQ ID NO: 3 - DNA, 24 bp, synthetic construct
  source 1..24: mol_type = other DNA
tctggttactgccagttgaatctg

SEQ ID NO: 4 - DNA, 20 bp, synthetic construct
  source 1..20: mol_type = other DNA
ttacaaacattggccgcaaa

SEQ ID NO: 5 - DNA, 18 bp, synthetic construct
  source 1..18: mol_type = other DNA
gcgcgacattccgaagaa